(19) United States
(12) Patent Application Publication
(10) Pub. No.: US 2015/0119717 A1
(43) Pub. Date: Apr. 30, 2015

(54) ULTRASONIC DEVICE, ULTRASONIC PROBE HEAD, ULTRASONIC PROBE, ELECTRONIC APPARATUS, AND ULTRASONIC IMAGING APPARATUS

(71) Applicant: SEIKO EPSON CORPORATION, Tokyo (JP)

(72) Inventors: Kazuki YOSHIDA, Fujimi-machi (JP); Yasunori ONISHI, Shiojiri (JP); Hironori SUZUKI, Chino (JP)

(21) Appl. No.: 14/515,012

(22) Filed: Oct. 15, 2014

(30) Foreign Application Priority Data
Oct. 28, 2013 (JP) 2013-223009

(51) Int. Cl. A61B 8/00; A61B 8/14; A61B 8/08; G10K 11/30

(52) U.S. Cl. CPC A61B 8/4444; G10K 11/30; A61B 8/145; A61B 8/4494; A61B 8/5207; A61B 8/461; A61B 8/4272; A61B 8/4427; A61B 8/4405
USPC 600/447; 600/472

(57) ABSTRACT

Provided is an ultrasonic device including: an ultrasonic element array substrate having a plurality of ultrasonic elements configured to perform at least one of transmission and reception of ultrasound; an acoustic lens configured to focus the ultrasound; an acoustic matching unit formed using resin, the acoustic matching unit being arranged between the ultrasonic element array substrate and the acoustic lens; and a plurality of columnar spacing members arranged between the ultrasonic element array substrate and the acoustic lens so as to be in contact with the ultrasonic element array substrate and the acoustic lens.

### BACKGROUND

1. Technical Field

The present invention relates to an ultrasonic device, an ultrasonic probe head, an ultrasonic probe, an electronic apparatus, and an ultrasonic imaging apparatus.

2. Related Art

Ultrasonic devices using ultrasonic elements that transmit and receive ultrasound have been used in various applications. JP-A-2011-35916, which is an example of related art, discloses an ultrasonic endoscope provided with ultrasonic elements. This ultrasonic endoscope is provided with ultrasonic elements of the electrostatic capacitance type that transmit and receive ultrasound, and an acoustic lens that focuses the ultrasound.

The ultrasonic elements apply an AC voltage to a substrate on which a lower electrode is installed and a membrane on which an upper electrode is installed. This causes an electrostatic force to act on the substrate and the membrane, so that the membrane vibrates so that ultrasound is transmitted. The ultrasound passes through the acoustic lens, thereby being emitted so as to be focused on a predetermined point. The acoustic lens is formed using silicone resin, which is a material that easily transfers ultrasound to a material being examined and that is easily deformed due to a stress being applied.

An acoustic lens transmits ultrasound more easily when it is in contact with the material being examined. Further, since the position of the acoustic lens is controlled by an operator, the acoustic lens may be pressed by the material being examined in some cases. In JP-A-2011-35916, the periphery of the acoustic lens is supported by a metal package. Accordingly, when stress is applied to the acoustic lens by the material being examined, the center of the acoustic lens is easily deformed because it is held by its outer circumference. When the acoustic lens is deformed, the point on which the ultrasound is focused is shifted, and the sound pressure at the point on which the ultrasound was to be focused is reduced. Therefore, an ultrasonic device capable of transmitting and receiving ultrasound efficiently by suppressing the deformation of the acoustic lens has been desired.

### SUMMARY

The invention has been devised to solve the aforementioned problems and can be practiced as embodiments or application examples described below.

### APPLICATION EXAMPLE 1

An ultrasonic device according to this application example includes: an ultrasonic element array substrate having a plurality of ultrasonic elements configured to perform at least one of transmission and reception of ultrasound; an acoustic lens configured to focus the ultrasound; an acoustic matching unit formed using resin, the acoustic matching unit being arranged between the ultrasonic element array substrate and the acoustic lens; and a plurality of columnar spacing members arranged between the ultrasonic element array substrate and the acoustic lens so as to be in contact with the ultrasonic element array substrate and the acoustic lens.

According to this application example, a plurality of ultrasonic elements are installed on the ultrasonic element array substrate. An ultrasonic element transmits or receives ultrasound. Alternatively, the ultrasonic element transmits and receives ultrasound. The ultrasound transmitted by the ultrasonic element passes through the acoustic matching unit and the acoustic lens to a material being examined. The acoustic matching unit adjusts the acoustic impedance between the acoustic lens and the ultrasonic element. This makes it difficult for ultrasound to be reflected by the interface between the acoustic lens and the ultrasonic element, and makes it difficult for ultrasound to be reflected by the interface between the acoustic matching unit and the acoustic lens. Accordingly, ultrasound is emitted efficiently to the material being examined.

The acoustic lens is used while in contact with the material being examined. At this time, the acoustic lens is pressed by the material being examined, and stress occurs inside the acoustic lens. The resin of the acoustic matching unit is susceptible to deformation, and therefore is deformed due to the stress of the acoustic lens. On the other hand, the columnar spacing members are in contact with the acoustic lens and the ultrasonic element array substrate, and transfer the stress of the acoustic lens to the ultrasonic element array substrate. Further, the thickness of the acoustic matching unit is kept constant, thereby suppressing the deformation of the acoustic lens so that ultrasound can be focused accurately. Further, ultrasound reflected by the material being examined also can be focused accurately on the ultrasonic element since the deformation of the acoustic lens is suppressed. As a result, the ultrasonic device can transmit and receive ultrasound efficiently.

### APPLICATION EXAMPLE 2

In the ultrasonic device according to the aforementioned application example, the spacing members are arranged in locations that do not overlap with the ultrasonic elements in plan view, as viewed in a thickness direction of the ultrasonic element array substrate.

According to this application example, the spacing members are arranged at locations that do not overlap with the ultrasonic elements. The ultrasonic elements are overlapped by the ultrasonic matching unit formed of resin. Accordingly, the ultrasonic device can emit ultrasound whose acoustic impedance has been adjusted by the acoustic matching unit. Further, in the ultrasonic device, the acoustic matching unit can adjust the acoustic impedance of the received ultrasound and emit it to the ultrasound element.

### APPLICATION EXAMPLE 3

In the ultrasonic device according to the aforementioned application example, the spacing members are arranged between the ultrasonic elements so as to extend in the form of a wall and hardly allow ultrasound to pass therethrough in an in-plane direction of the ultrasonic element array substrate.

According to the present example, the spacing members are arranged between the ultrasonic elements so as to extend in the form of a wall. The spacing members make it difficult for ultrasound to pass therethrough in the in-plane direction of the ultrasonic element array substrate and regulate the direction in which the ultrasound propagates. Note that the in-plane direction of the ultrasonic element array substrate is a direction parallel to the surface of ultrasonic element array substrate. Accordingly, it is possible to suppress a case in which ultrasonic elements located with a spacing member interposed therebetween influence each other when receiving and transmitting ultrasound.

### APPLICATION EXAMPLE 4

In the ultrasonic device according to the aforementioned application example, the spacing members have lower water permeability than the acoustic matching unit and are arranged so as to cover wiring for transmitting an electrical signal to the ultrasonic elements.

According to this application example, the spacing members are arranged so as to cover the wiring. The spacing members are sites that have low water permeability and hardly allow moisture to pass therethrough. Accordingly, the spacing members suppress a case in which moisture attaches to the wiring, and therefore it is possible to prevent galvanic corrosion of the wiring.

### APPLICATION EXAMPLE 5

In the ultrasonic device according to the aforementioned application example, a flow path through which material for the acoustic matching unit flows is formed between two of the spacing members.

According to this application example, the spacing members extend in the form of a wall. When the acoustic matching unit is to be formed, a flow path through which the material for the acoustic matching unit flows is formed between two spacing members. The material for the acoustic matching unit flows along the spacing members, and therefore the spaces between the spacing members can be filled tightly with the material for the acoustic matching unit.

### APPLICATION EXAMPLE 6

In the ultrasonic device according to the aforementioned application example, the shape of the spacing members is circular or elliptical in plan view.

According to this application example, the shape of the spacing members is circular or elliptical. A circle or an ellipse has no corners, thereby allowing a fluid to flow along its outer circumference with little resistance. Accordingly, when the material for the acoustic matching unit is allowed to flow in the location where the spacing members are present, the material for the acoustic matching unit moves along the spacing members. At this time, the material for the acoustic matching unit pushes out the air located in the space for the acoustic matching unit, and therefore the intervals between the spacing members can be filled tightly with the material for the acoustic matching unit.

### APPLICATION EXAMPLE 7

An ultrasonic probe head according to this application example includes: the ultrasonic device according to one of the aforementioned examples; and a housing configured to support the ultrasonic device.

According to this application example, the ultrasonic probe head includes the aforementioned ultrasonic device and the housing configured to support the ultrasonic device. The ultrasonic probe head of this application example includes the ultrasonic device that appropriately maintains the thickness of the acoustic matching unit, and that transmits and receives ultrasound efficiently. Accordingly, it is possible to provide the ultrasonic probe head that transmits and receives ultrasound efficiently.

### APPLICATION EXAMPLE 8

An ultrasonic probe according to this application example includes: the ultrasonic device according to one of the aforementioned examples; and a driving circuit configured to drive the ultrasonic device.

According to this application example, the ultrasonic probe includes the aforementioned ultrasonic device and the driving circuit configured to drive the ultrasonic device. The ultrasonic probe of this application example includes the ultrasonic device that appropriately maintains the thickness of the acoustic matching unit, and that transmits and receives ultrasound efficiently. Accordingly, it is possible to provide the ultrasonic probe that transmits and receives ultrasound efficiently.

### APPLICATION EXAMPLE 9

An electronic apparatus according to this application example includes: the ultrasonic device according to one of the aforementioned examples; and a processing unit connected to the ultrasonic device, the processing unit being configured to generate an image using an output of the ultrasonic device.

According to this application example, the electronic apparatus includes the aforementioned ultrasonic device and the processing unit. The processing unit generates image data using the output of the ultrasonic device. The electronic apparatus of this application example includes the ultrasonic device that appropriately maintains the thickness of the acoustic matching unit, and that transmits and receives ultrasound efficiently. Accordingly, it is possible to provide the electronic apparatus that transmits and receives ultrasound efficiently.

### APPLICATION EXAMPLE 10

An ultrasonic imaging apparatus according to this application example includes: the ultrasonic device according to one of the aforementioned examples; and a processing unit connected to the ultrasonic device, the processing unit being configured to perform processing to generate an image using an output of the ultrasonic device; and a display unit configured to display the image.

According to this application example, the ultrasonic imaging apparatus includes the aforementioned ultrasonic device, the processing unit, and the display unit. The processing unit generates image data using the output of the ultrasonic device. The display unit displays images generated by the processing unit. The ultrasonic imaging apparatus of this application example includes the ultrasonic device that appropriately maintains the thickness of the acoustic matching unit, and that transmits and receives ultrasound efficiently. Accordingly, it is possible to provide the ultrasonic imaging apparatus that transmits and receives ultrasound efficiently.

### BRIEF DESCRIPTION OF THE DRAWINGS

The invention will be described with reference to the accompanying drawings, wherein like numbers reference like elements.

FIG. 1 is a schematic perspective view showing a configuration of an ultrasonic imaging apparatus.

FIG. 2 is a schematic side cross-sectional view showing a part of a structure of an ultrasonic probe.

FIG. 3 is a schematic cross-sectional view showing a main part of the structure of the ultrasonic probe.

FIG. 4 is a block diagram illustrating the control of the ultrasonic imaging apparatus.

FIG. 5 is a schematic plan view showing a structure of an ultrasonic device.

FIGS. 6A and 6C are schematic side cross-sectional views showing the structure of the ultrasonic device, FIGS. 6B and 6D are schematic side views showing the structure of the ultrasonic device.

FIG. 7A is a schematic plan view showing a configuration of an ultrasonic element, and FIG. 7B is a schematic side cross-sectional view showing the configuration of the ultrasonic element.

FIG. 8 is a schematic plan view showing a configuration of an ultrasonic element array substrate.

FIG. 9 is a flow chart of a method for manufacturing an ultrasonic device.

FIGS. 10A to 10E are schematic diagrams for describing the method for manufacturing an ultrasonic device.

FIGS. 11A to 11D are schematic diagrams for describing the method for manufacturing an ultrasonic device.

FIG. 12A is a schematic plan view showing a main part of a configuration of an ultrasonic element, and FIG. 12B is a schematic plan view showing a configuration of an ultrasonic element array substrate.

FIGS. 13A and 13B are schematic side views showing a configuration of an ultrasonic probe.

FIG. 14 is a schematic perspective view showing a configuration of an ultrasonic imaging apparatus.

### DESCRIPTION OF EXEMPLARY EMBODIMENTS

In this embodiment, characteristic examples of an an ultrasonic device and an ultrasonic probe and an ultrasonic imaging apparatus provided with the ultrasonic device will be described with reference to the drawings. It should be noted that the sizes of the members in the drawings are scaled differently in each figure so as to be perceptible.

### First Embodiment

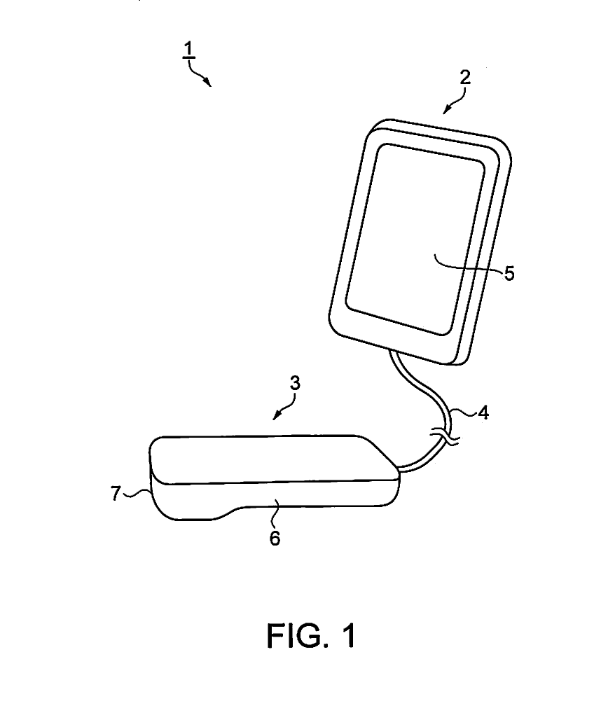

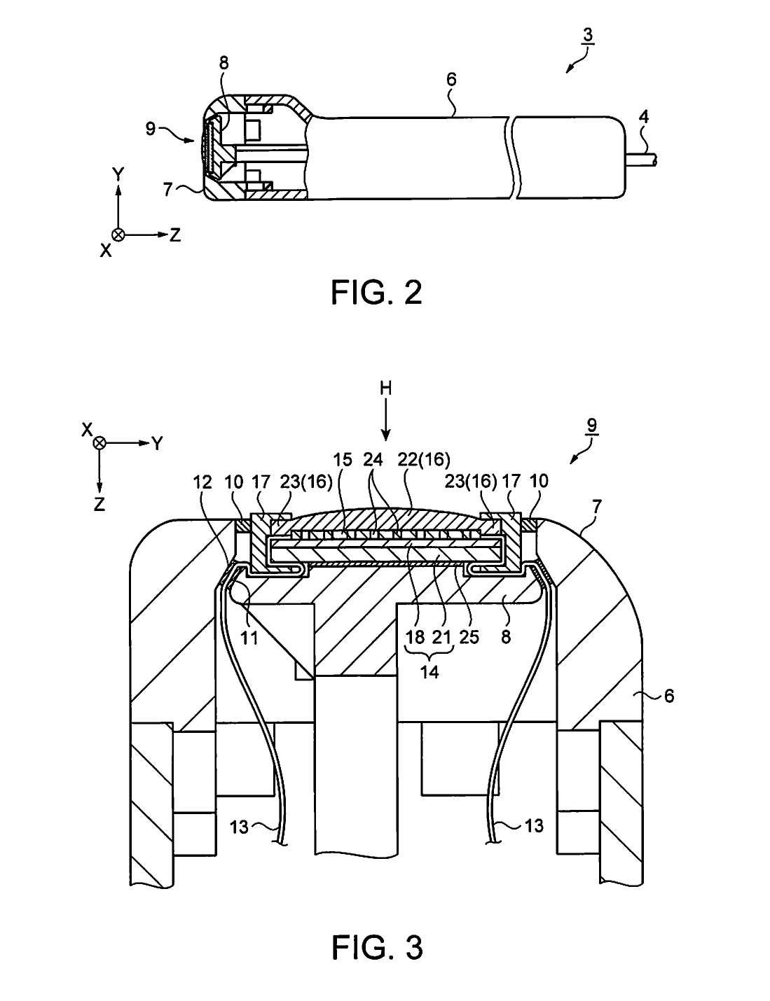

In this embodiment, an ultrasonic imaging apparatus for examining an interior portion of a human body will be described as an example of an electronic apparatus with reference to FIG. 1 to FIG. 11D. FIG. 1 is a schematic perspective view showing a configuration of the ultrasonic imaging apparatus. FIG. 2 is a schematic side cross-sectional view showing a part of a structure of an ultrasonic probe. FIG. 3 is a schematic cross-sectional view showing a main part of the structure of the ultrasonic probe.

As shown in FIG. 1, an ultrasonic imaging apparatus 1 serving as an electronic apparatus includes an apparatus body 2 and an ultrasonic probe 3. The apparatus body 2 and the ultrasonic probe 3 are connected to each other by a cable 4. The apparatus body 2 and the ultrasonic probe 3 can exchange electrical signals via the cable 4. The apparatus body 2 incorporates a display unit 5 such as a display panel. The display unit 5 is a touch panel display, and serves also as a user interface unit with which an operator inputs information into the apparatus body 2. Hereinafter, the user interface unit will be referred to as “UI unit”.

In the apparatus body 2, an image is generated on the basis of ultrasound detected by the ultrasonic probe 3, and the detection results that are output as an image are displayed on the screen of the display unit 5. The ultrasonic probe 3 includes a rectangular parallelepiped housing 6. The cable 4 is connected to one end in the longitudinal direction of the housing 6. On the opposite side, a head portion 7 that transmits and receives ultrasound is provided. It should be noted that a configuration of the ultrasonic imaging apparatus 1 is used in which the apparatus body 2 and the ultrasonic probe 3 are connected by the cable 4. A configuration is also possible in which the apparatus body 2 and the ultrasonic probe 3 wirelessly exchange signals without using the cable 4.

As shown in FIG. 2, the ultrasonic probe 3 includes an ultrasonic device 9 that is fixed to a support member 8 and that is accommodated within the housing 6. The ultrasonic device 9 is exposed from the head portion 7 of the housing 6 so that ultrasound is output from the ultrasonic device 9 to a target object. Further, the ultrasonic device 9 receives reflected waves of the ultrasound from the object. Such reflected waves are referred to also as echo waves. The housing 6 has a cylindrical shape, which is easy for the operator to grip. The ultrasonic device 9 is installed at one end of the housing 6, and the cable 4 is installed at the other end thereof. A direction extending from the ultrasonic device 9 toward the cable 4 is referred to as Z direction. The two directions orthogonal to the Z direction are referred to as the X direction and the Y direction. The ultrasonic device 9 is approximately plate-shaped and extends in the X direction and the Y direction. The ultrasonic device 9 is longer in the X direction than in the Y direction.

As shown in FIG. 3, there is a gap between the ultrasonic device 9 and the head portion 7 of the housing 6. A sealing portion 10 filled with a silicone-based sealing material is provided in the gap. This sealing portion 10 prevents moisture, etc., from entering the ultrasonic device 9 in the housing 6 of the ultrasonic probe 3. The support member 8 is located on the Z direction side of the ultrasonic device 9. A sealing structure is installed between the support member 8 and the head portion 7. This sealing structure includes an adhesive member 11 and an adhesive member 12. The adhesive member 11 is a member, such as a double-sided adhesive tape having elasticity, which is attached to the outer circumferential portion of the support member 8. The adhesive member 12 is a member, such as a double-sided adhesive tape having elasticity, which is attached to the housing 6.

An FPC 13 (Flexible Printed Circuit) that connects the ultrasonic device 9 to a processing circuit is interposed between the adhesive member 11 and the adhesive member 12. The FPC 13 is fixed by being sandwiched by the adhesive member 11 and the adhesive member 12. The FPC 13 is referred to also as flexible printed circuit board. As the adhesive member 11 and the adhesive member 12, a double-sided adhesive tape formed by applying an acrylic-based adhesive material to a closed cell foam material such as polyethylene or urethane can be used, for example. In this way, a double sealing structure is employed for the ultrasonic probe 3, in which the sealing portion 10, the adhesive member 11, and the adhesive member 12 prevent moisture, dust, and the like from entering the inside of the housing 6.

The ultrasonic device 9 includes an ultrasonic element array substrate 14, an acoustic matching unit 15, an acoustic lens 16, the FPC 13, and a frame 17 as a fixing frame. The ultrasonic element array substrate 14 has an element substrate 18 and a back plate 21. The element substrate 18 is a substrate on which a plurality of ultrasonic elements are arranged in an array on a surface on the −Z direction side, and has a rectangular shape elongated in the X direction, in plan view as viewed from the Z direction. The element substrate 18 is formed using a silicon substrate and has a thickness of about 150 μm to 200 μm. The back plate 21 having the same flat plate shape as the element substrate 18 is adhered to the opposite surface of the element-formed surface of the element substrate 18 oriented in the −Z direction. The back plate 21 serves to suppress excess vibration of the element substrate 18 and absorb ultrasound. A silicon substrate with a thickness of 500 μm to 600 μm is used for the back plate 21. For the back plate 21, a metal plate may be used, rather than such a silicon substrate. In the case where the influence of ultrasound that travels in the Z direction from the element substrate 18 is small, the ultrasonic device 9 may be formed without using the back plate 21.

On the surface of the element substrate 18 on which the ultrasonic elements are formed, a plurality of terminals connected to the plurality of ultrasonic elements are installed along the long edge extending in the X direction, in plan view. These terminals are connected to the terminals of the FPC 13 and are thus connected electrically as well.

On the surface of the element substrate 18 on which the ultrasonic elements are formed, the acoustic lens 16 is arranged. The planar shape of the acoustic lens 16 as viewed from the −Z direction is the same as the shape of the ultrasonic element array substrate 14. On the acoustic lens 16, a lens portion 22 is provided. The lens portion 22 has a surface facing the −Z direction that protrudes in the thickness direction with a predetermined curvature. On the surface facing the Z direction, a wall 23 that projects in the thickness direction and that is formed on the outer edge portion of the acoustic lens 16 is provided. The acoustic lens 16 is formed using a resin such as silicone resin. It is possible to adjust the acoustic impedance of the silicone resin by adding silica, or the like, to the silicone resin to change the specific gravity of the silicone resin.

The acoustic matching unit 15 is formed between the ultrasonic element array substrate 14 and the acoustic lens 16. For the acoustic matching unit 15, a silicone-based adhesive material is used. Curing of the adhesive material causes the ultrasonic element array substrate 14 and the acoustic lens 16 to be secured (adhered) to each other. The thus cured adhesive material (resin) functions as the acoustic matching unit 15. A plurality of columnar spacing members 24 are installed in parallel with the acoustic matching unit 15. The spacing members 24 keep the thickness of the acoustic matching unit 15 constant. When the acoustic lens 16 is pressed by a target object, the spacing members 24 transfer the force applied onto the acoustic lens 16 to the ultrasonic element array substrate 14. The spacing members 24 suppress the deformation of the acoustic lens 16 due to a reaction force received from the ultrasonic element array substrate 14.

The acoustic lens 16 serves to focus ultrasound transmitted from the ultrasonic elements of the element substrate 18 and guide them efficiently to a target object. The acoustic lens 16 also serves to guide echo waves reflected back from the object efficiently to the ultrasonic elements. The acoustic matching unit 15 serves to relax the acoustic impedance mismatch between the acoustic lens 16 and the ultrasonic elements. The back plate 21 of the ultrasonic device 9 is fixed to the support member 8 by an adhesive material 25.

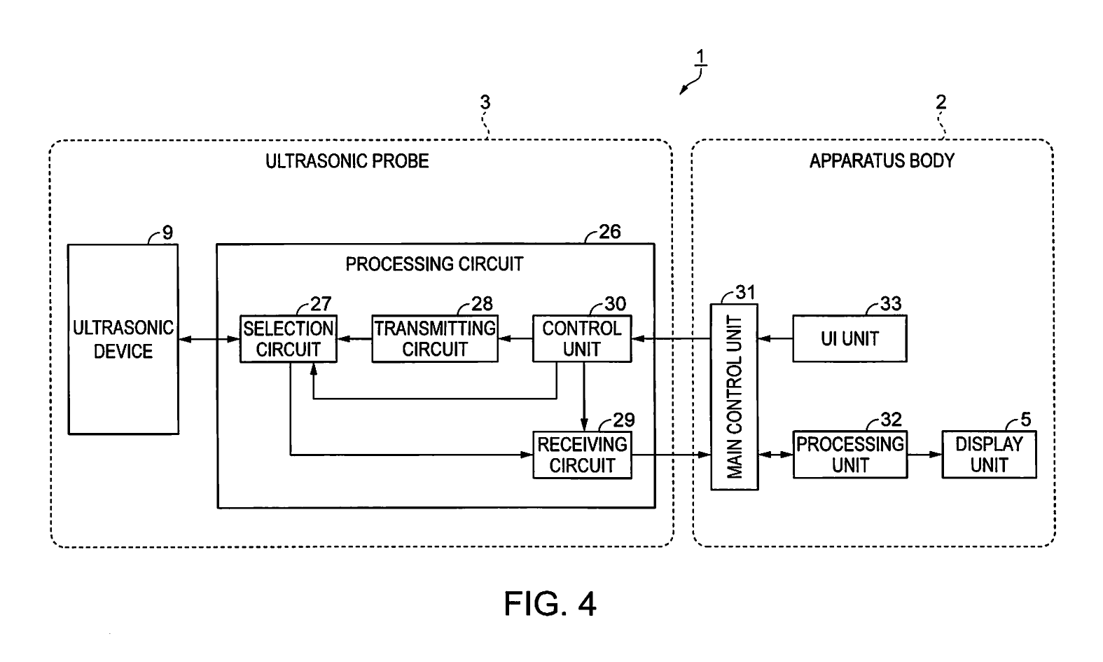

FIG. 4 is a block diagram illustrating the control of the ultrasonic imaging apparatus. As shown in FIG. 4, the ultrasonic imaging apparatus includes the apparatus body 2 and the ultrasonic probe 3. The ultrasonic probe 3 includes the ultrasonic device 9 and a processing circuit 26 as a driving circuit. The processing circuit 26 has a selection circuit 27, a transmitting circuit 28, a receiving circuit 29, and a control unit 30. This processing circuit 26 performs transmission processing and reception processing for the ultrasonic device 9.

The transmitting circuit 28 outputs transmission signals VT to the ultrasonic device 9 via the selection circuit 27 in a transmission period. Specifically, the transmitting circuit 28 generates the transmission signals VT, on the basis of control by the control unit 30, and outputs them to the selection circuit 27. Then, the selection circuit 27 outputs the transmission signals VT from the transmitting circuit 28, on the basis of control by the control unit 30. The frequency and amplitude voltage of the transmission signals VT are set by the control unit 30.

The receiving circuit 29 performs reception processing to receive reception signals VR from the ultrasonic device 9. Specifically, the receiving circuit 29 receives the reception signals VR from the ultrasonic device 9 via the selection circuit 27 in a reception period. The receiving circuit 29 performs reception processing such as amplification of the reception signals, gain setting, frequency setting, and A/D conversion (analog/digital conversion). The receiving circuit 29 outputs the results of the reception processing to the apparatus body 2 as detected data (detected information). The receiving circuit 29, for example, can be composed of a low-noise amplifier, a voltage-controlled attenuator, a programmable gain amplifier, a low-pass filter, an A/D converter, and the like.

The control unit 30 controls the transmitting circuit 28 and the receiving circuit 29. Specifically, the control unit 30 controls the transmitting circuit 28 for generation of the transmission signals VT and output processing, and controls the receiving circuit 29 for frequency setting of the reception signals VR, gain, or the like. The selection circuit 27 outputs the selected transmission signals VT to the ultrasonic device 9, on the basis of control by the control unit 30.

The apparatus body 2 includes the display unit 5, a main control unit 31, a processing unit 32, and a UI unit 33 (user interface unit). The main control unit 31 controls the ultrasonic probe 3 for transmission and reception of ultrasound, and controls the processing unit 32 for image processing of detected data, for example. The processing unit 32 receives detected data from the receiving circuit 29, and performs image processing to remove noises, generation of image data to be displayed, or the like. The UI unit 33 includes a function of receiving input of a user instruction, and the UI unit 33 outputs necessary instruction (command) to the main control unit 31 on the basis of operation (such as touch panel operation) by the user. The display unit 5, for example, is a liquid crystal display, and receives input of the image data to be displayed from the processing unit 32 and displays it. It should be noted that part of control by the main control unit 31 may be performed by the control unit 30 of the processing circuit 26, or part of control by the control unit 30 may be performed by the main control unit 31.

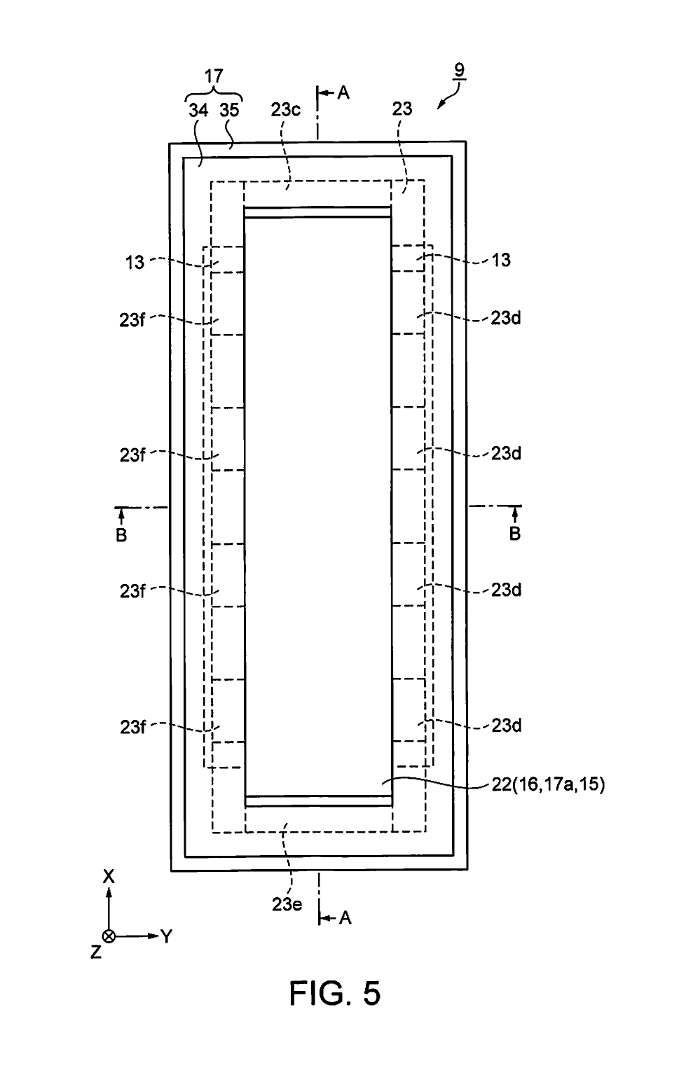

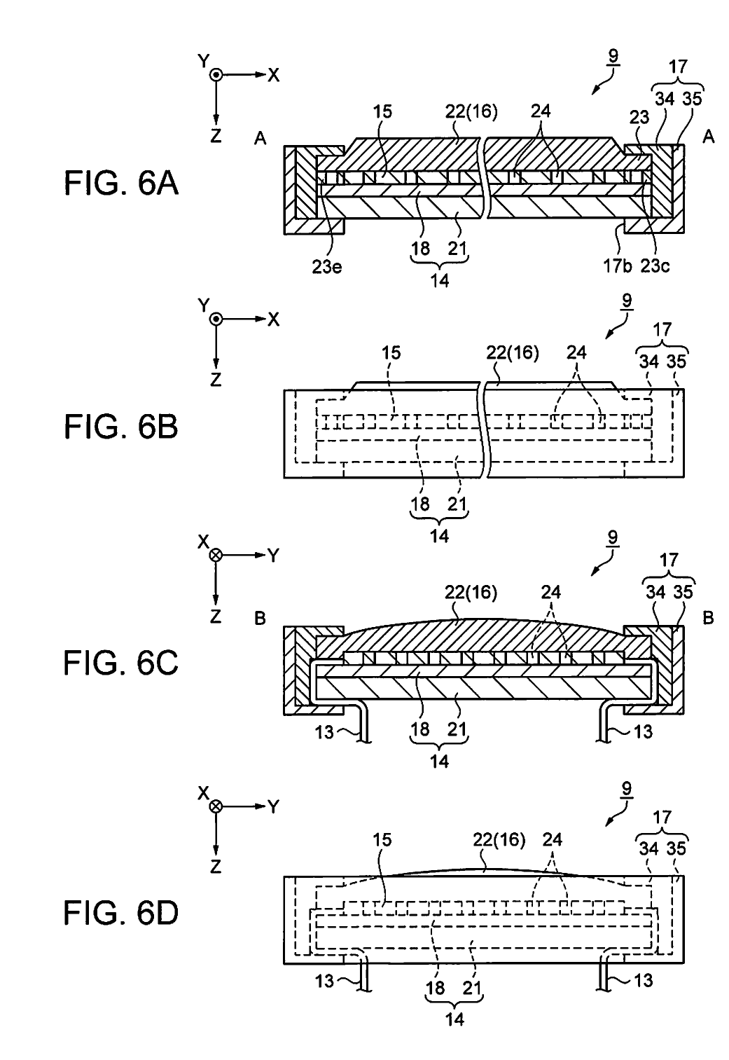

FIG. 5 is a schematic plan view showing a structure of the ultrasonic device of the ultrasonic probe 3, as viewed in the direction of the arrow H in FIG. 3. FIG. 6A is a schematic side cross-sectional view showing the structure of the ultrasonic device, which is taken along line A-A in FIG. 5. FIG. 6B is a schematic side view showing the structure of the ultrasonic device, as viewed from the Y direction. FIG. 6C is a schematic side cross-sectional view showing the structure of the ultrasonic device, which is taken along line B-B in FIG. 5. FIG. 6D is a schematic side view showing the structure of the ultrasonic device, as viewed from the −X direction.

As shown in FIG. 5 and FIGS. 6A to 6D, the ultrasonic device 9 has a rectangular parallelepiped shape elongated in the X direction. When the ultrasonic device 9 is viewed from the −Z direction, the frame 17 has a rectangular first hole 17a formed at its center, and the lens portion 22 is exposed through the first hole 17a. When the ultrasonic device 9 is viewed from the Z direction, the frame 17 has a rectangular second hole 17b formed at its center, and the back plate 21 is exposed through the second hole 17b.

The frame 17 is composed of an inner frame 34 located on the inner side and an outer frame 35 located on the outer side. The inner frame 34 presses the acoustic lens 16 from the −Z direction side. The outer frame 35 presses the ultrasonic element array substrate 14 from the Z direction side. The inner frame 34 and the outer frame 35 are adhered to each other so as to be secured. Accordingly, the frame 17 fixes the ultrasonic element array substrate 14, the acoustic matching unit 15, and the acoustic lens 16 by sandwiching them in the Z direction.

The spacing members 24 are installed in parallel with the acoustic matching unit 15. The spacing members 24 are arranged between the ultrasonic element array substrate 14 and the acoustic lens 16 that are sandwiched by the frame 17. The frame 17 reliably fixes the ultrasonic element array substrate 14 and the acoustic lens 16 by sandwiching them with the spacing members 24 interposed therebetween. Accordingly, the spacing members 24 can keep the thickness of the acoustic matching unit 15 constant.

A first recessed portion 23c is formed in the X direction of the wall 23, and a third recessed portion 23e is formed in the −X direction thereof. The first recessed portion 23c and the third recessed portion 23e are continuous with the acoustic matching unit 15 in a location opposing the lens unit 22. The acoustic matching unit 15 is located also inside the first recessed portion 23c and the third recessed portion 23e.

Second recessed portions 23d are formed in the Y direction of the wall 23, and fourth recessed portions 23f are formed on the −Y direction thereof. The second recessed portions 23d and the fourth recessed portions 23f are continuous with the acoustic matching unit 15 at a location opposing the lens 22. The acoustic matching unit 15 is located also inside the second recessed portions 23d and the fourth recessed portions 23f.

The spacing members 24 are located in the first recessed portion 23c and the third recessed portion 23e. The spacing members 24 are arranged between the ultrasonic element array substrate 14 and the acoustic lens 16 in a portion sandwiched by the frame 17 in plan view, as viewed from the −Z direction. The frame 17 sandwiches the ultrasonic element array substrate 14 and the acoustic lens 16 with the spacing members 24 interposed therebetween, and therefore the spacing members 24 can reliably keep the thickness of the acoustic matching unit 15 constant.

The FPC 13 is sandwiched by the ultrasonic element array substrate 14 and the wall 23 on the Y direction side and the −Y direction side of the acoustic lens 16. The frame 17 holds the ultrasonic element array substrate 14 and the wall 23 by sandwiching them, thereby preventing the FPC 13 from lifting in a portion where the ultrasonic element array substrate 14 and the FPC 13 are connected to each other. Thus, the FPC 13 is reliably fixed.

Letting X be the wavelength of the ultrasound to be used, the thickness of the acoustic matching unit 15 is set to an odd multiple of ¼λ. The thickness of the spacing members 24 in the Z direction is set equal to the thickness of the acoustic matching unit 15.

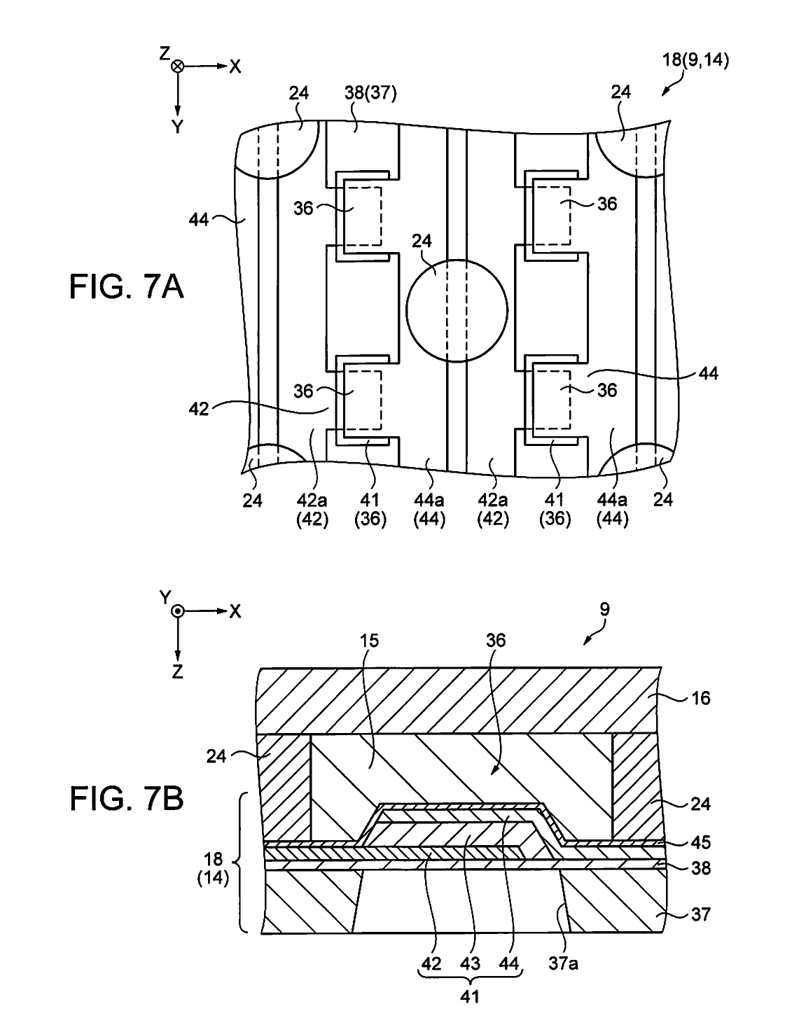

FIG. 7A is a schematic plan view showing a configuration of an ultrasonic element, with the acoustic lens 16 and the acoustic matching unit 15 omitted and with the spacing members 24 installed. FIG. 7B is a schematic side cross-sectional view showing the configuration of the ultrasonic element, with the acoustic lens 16 and the acoustic matching unit 15 installed. As shown in FIGS. 7A and 7B, a plurality of ultrasonic elements 36 are installed in the element substrate 18. An ultrasonic element 36 has a base substrate 37 as a substrate, a vibrating membrane 38 (membrane) formed on the base substrate 37, and a piezoelectric body 41 provided on the vibrating membrane 38. The piezoelectric body 41 has a first electrode 42 serving as a lower electrode, a piezoelectric layer 43, and a second electrode 44 serving as an upper electrode.

An opening 37a is formed in the base substrate 37 made of a silicon substrate, or the like, and the ultrasonic element 36 includes the vibrating membrane 38 that covers the opening 37a so as to close it. The vibrating membrane 38 is composed of a double layer structure, for example, of a SiO₂ layer and a ZrO₂ layer. In the case where the base substrate 37 is a silicon substrate, the SiO₂ layer can be formed by subjecting the surface of the substrate to a thermal oxidation treatment. Further, the ZrO₂ layer can be formed on the SiO₂ layer, for example, by a technique such as sputtering. For example, in the case of using PZT (lead zirconate titanate) as the piezoelectric layer 43, the ZrO₂ layer is a layer for preventing Pb that constitutes the PZT from diffusing into the SiO₂ layer. Further, the ZrO₂ layer also has an effect of improving the warpage efficiency corresponding to distortion of the piezoelectric layer, etc.

The first electrode 42 is formed on the vibrating membrane 38. The piezoelectric layer 43 is formed on the first electrode 42. The second electrode 44 is formed further on the piezoelectric layer 43. That is, the piezoelectric body 41 has a structure in which the piezoelectric layer 43 is sandwiched between the first electrode 42 and the second electrode 44.

The first electrode 42 is formed of a thin metal film, and extends in the Y direction. A portion thereof protrudes in the X direction at the ultrasonic element 36. The first electrode 42 is arranged over a plurality of piezoelectric bodies 41, and functions also as wiring. The portion of the first electrode 42 that functions as wiring will be referred to as “first line 42a”. The piezoelectric layer 43 is formed, for example, of a thin PZT (lead zirconate titanate) film, and is provided to cover part of the first electrode 42. It should be noted that the material of the piezoelectric layer 43 is not limited to PZT. For example, lead titanate (PbTiO₃), lead zirconate (PbZrO₃), lead lanthanum titanate ((Pb, La) TiO₃), or the like, may be used. The second electrode 44 is formed of a thin metal film, and is provided to cover the piezoelectric layer 43. The second electrode 44 extends in the Y direction, and a portion thereof protrudes in the −X direction at the ultrasonic element 36. The second electrode 44 is arranged over the plurality of piezoelectric bodies 41, and functions also as wiring. The portion of the second electrode 44a that functions as wiring will be referred to as “second line 44a”.

When the element substrate 18 is viewed from the −Z direction, the first electrode 42 and the second electrode 44 overlap each other at the ultrasonic element 36. The first line 42a and the second line 44a are portions at which the first electrode 42 and the second electrode 44 do not overlap. The spacing members 24 are arranged in locations where the first line 42a and the second line 44a are installed. The spacing members 24 are installed in locations that do not overlap with the ultrasonic elements 36. The acoustic matching unit 15 is installed so as to overlap the ultrasonic elements 36. The spacing members 24 do not need to be arranged in all locations that do not overlap with the ultrasonic elements 36, and it is sufficient to provide the spacing members 24 in some locations. The spacing members 24 may be provided in an amount such that the thickness of the acoustic matching unit 15 can be kept constant.

An insulation film 45 that prevents moisture permeation from the outside and insulates the acoustic matching unit 15 from the first electrode 42 and the second electrode 44 is provided to cover the ultrasonic element 36. The insulation film 45 is formed of a material such as alumina, and is provided entirely or partially on the surface of the ultrasonic element 36. Further, the insulation film 45 is arranged to cover the first electrode 42 and the second electrode 44.

The piezoelectric layer 43 expands and contracts in the in-plane direction due to a voltage applied between the first electrode 42 and the second electrode 44. Accordingly, when a voltage is applied to the piezoelectric layer 43, convex warpage occurs on the opening 37a side, so that the vibrating membrane 38 is deflected. Application of an AC voltage to the piezoelectric layer 43 causes the vibrating membrane 38 to vibrate in the membrane thickness direction, and the vibration of the vibrating membrane 38 causes ultrasound to be emitted from the opening 37a. The voltage (drive voltage) to be applied to the piezoelectric layer 43, for example, is 10 to 30 V from peak to peak, and the frequency thereof, for example, is 1 to 10 MHz.

The ultrasonic element 36 acts also as a receiving element to receive ultrasonic echo of the emitted ultrasound that is reflected by the target object and returns back. The ultrasonic echo vibrates the vibrating membrane 38, and stress is applied to the piezoelectric layer 43 due to this vibration, thereby generating a voltage between the first electrode 42 and the second electrode 44. This voltage can be output as a reception signal.

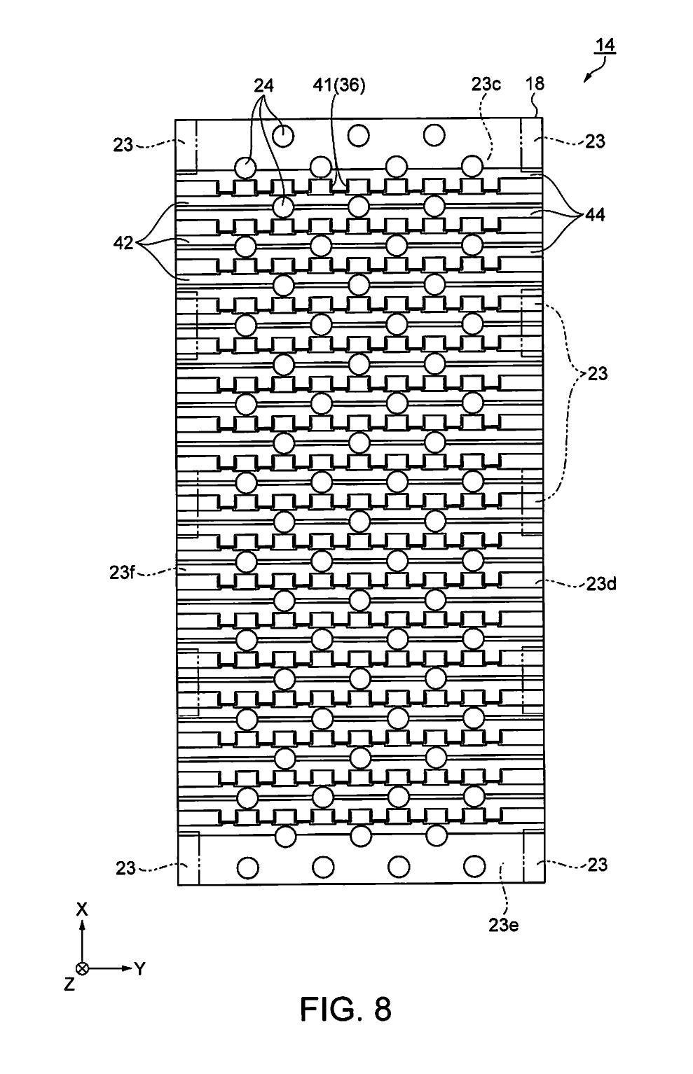

FIG. 8 is a schematic plan view showing a configuration of the ultrasonic element array substrate. As shown in FIG. 8, a plurality of ultrasonic elements 36 arranged in a matrix, the first electrode 42, and the second electrode 44 are installed in the ultrasonic element array substrate 14. For ease of viewing the figure, the ultrasonic elements 36 are arranged in 17 rows and 8 columns. However, there is no specific limitation on the number of rows and the number of columns.

During the transmission period in which ultrasound is emitted, the transmission signals VT output by the processing circuit 26 are supplied to the respective ultrasonic elements 36 via the second electrode 44. Meanwhile, during the reception period in which ultrasonic echo signals are received, the reception signals VR from the ultrasonic elements 36 are output to the processing circuit 26 via the second electrode 44. The first electrode 42 is supplied with a common voltage VCOM. It is sufficient that this common voltage is a constant voltage, and it need not be 0 V, or in other words, a ground potential. In the transmission period, a voltage that is the difference between the transmission signal voltage and the common voltage is applied to each of the ultrasonic elements 36, and ultrasound is emitted at a predetermined frequency.

The spacing members 24 are installed in the location where the first recessed portion 23c of the acoustic lens 16 is located, along an edge of the element substrate 18 on the X direction side. Similarly, the spacing members 24 are installed also in the location where the third recessed portion 23e of the acoustic lens 16 is located, along an edge of the element substrate 18 on the—X direction side. When the frame 17 sandwiches the acoustic lens 16 and the ultrasonic element array substrate 14, the spacing members 24 receive the load in a portion close to the frame 17, which enables the thickness of the acoustic matching unit 15 to be kept constant.

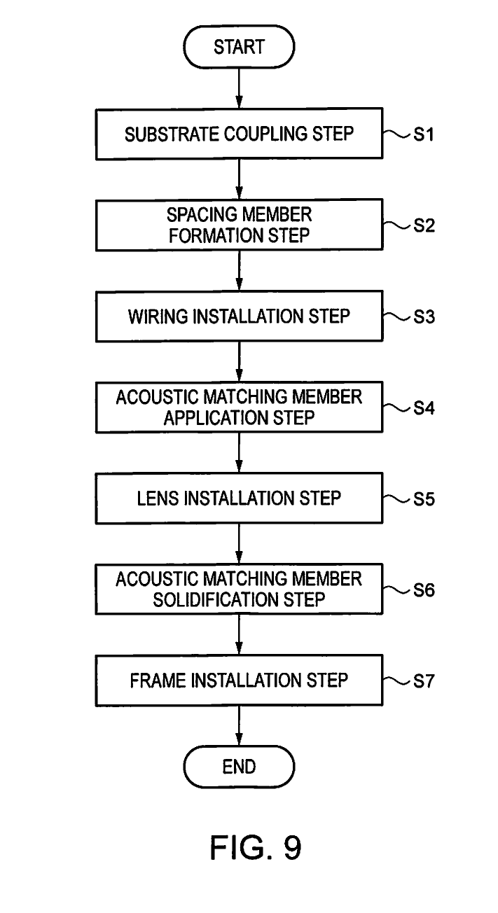

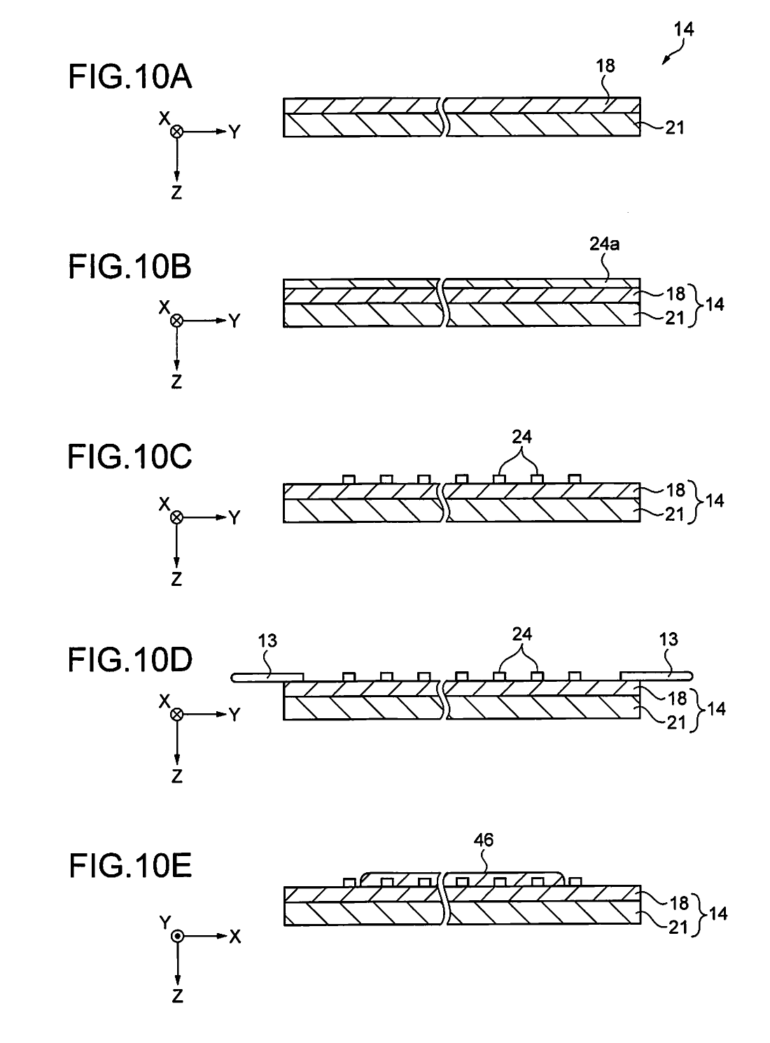

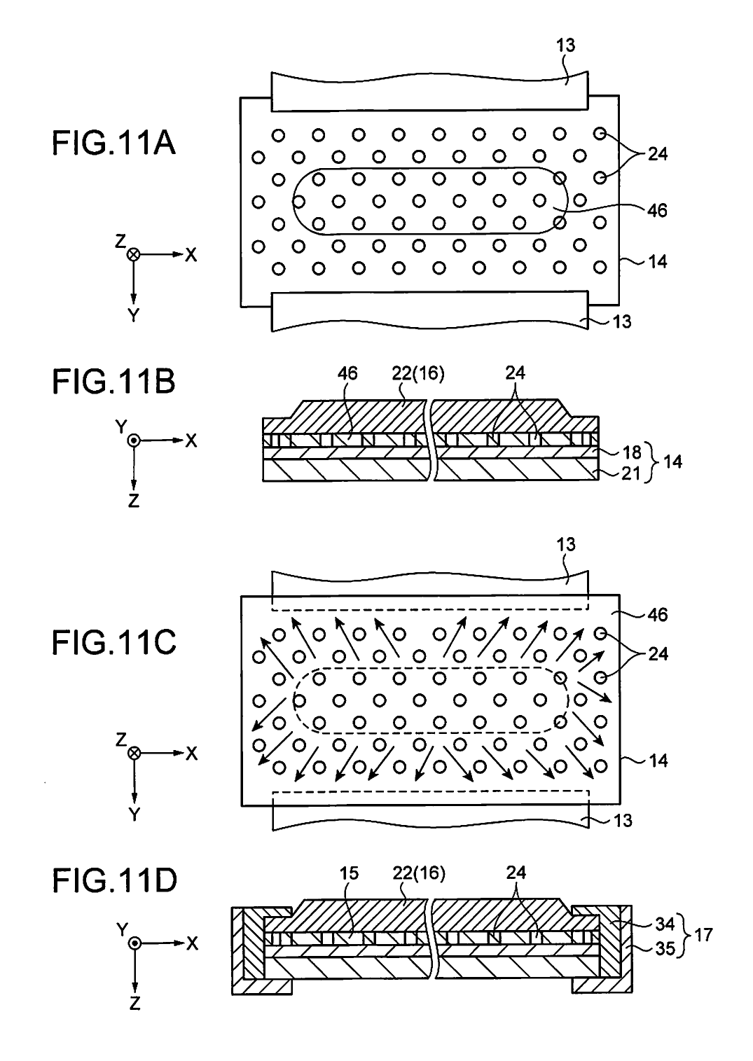

Next, a method for manufacturing the aforementioned ultrasonic device 9 will be described with reference to FIGS. 9 to 11. FIG. 9 is a flowchart of the method for manufacturing the ultrasonic device. FIGS. 10A to 10E and FIGS. 11A to 11D are schematic diagrams for describing the method for manufacturing the ultrasonic device. In the flowchart of FIG. 9, step S1 corresponds to a substrate coupling step. In this step, the element substrate 18 and the back plate 21 are coupled to each other so that the ultrasonic element array substrate 14 is formed. Next, the process proceeds to step S2. Step S2 corresponds to a spacing member formation step. In this step, the spacing members 24 are installed in the ultrasonic array substrate 14. Step S3 corresponds to a wiring installation step. In this step, the FPC 13 is coupled to the ultrasonic element array substrate 14. Next, the process proceeds to step S4. Step S4 corresponds to an acoustic matching unit application step. In this step, the material for the acoustic matching unit is applied and the acoustic matching unit is installed in the ultrasonic element array substrate 14. Next, the process proceeds to step S5.

Step S5 corresponds to a lens installation step. In this step, the acoustic lens 16 is installed so as to overlap the ultrasonic element array substrate 14. Next, the process proceeds to step S6. Step S6 corresponds to an acoustic matching unit solidification step. In this step, the acoustic matching unit is solidified. Next, the process proceeds to step S7. Step S7 corresponds to a frame installation step. In this step, the frame 17 is installed so as to sandwich the ultrasonic element array substrate 14 and the acoustic lens 16. By performing the aforementioned steps, the ultrasonic device 9 is achieved.

Next, with reference to FIGS. 10A to 10E and FIGS. 11A to 11D, the manufacturing method will be described in detail in correspondence with the steps shown in FIG. 9. FIG. 10A is a view corresponding to the substrate coupling step of step S1. As shown in FIG. 10A, the element substrate 18 and the back plate 21 are prepared in step S1. In the element substrate 18, the piezoelectric body 41 is formed. Since the method for manufacturing the piezoelectric body 41 is known to the public, the description thereof is omitted. An adhesive material is applied to the element substrate 18 or the back plate 21, and the element substrate 18 and the back plate 21 are laminated together. Next, the adhesive material is solidified by heating and drying, and the ultrasonic element array substrate 14 is complete.

FIG. 10B and FIG. 10C are views corresponding to the spacing member forming step of step S2. As shown in FIG. 10B, a spacing member film 24a is installed on the element substrate 18 in step S2. For the spacing member film 24a, a photosensitive resin film can be used. Then, an adhesive material is applied to the ultrasonic element array substrate 14, and the spacing member film 24a is adhered to the ultrasonic element array substrate 14. Next, the spacing member film 24a is masked by a predetermined pattern and is exposed to light. Subsequently, the spacing member film 24a is etched. As a result, the spacing members 24 are installed on the ultrasonic element array substrate 14, as shown in FIG. 10C. It is also possible to use a method different from the method with which the spacing member film 24a is adhered on the ultrasonic element array substrate 14. For example, the material of the spacing member film 24a may be applied using a method such as spin coating and dipping, followed by drying. For the material of the spacing member film 24a, epoxy resin can be used.

FIG. 10D is a view corresponding to the wiring installation step of step S3. As shown in FIG. 10D, the FPC 13 is prepared in step S3. Solder plating is applied to the ends of the wiring of the FPC 13. The first electrode 42 and the second electrode 44 extend to the ends on the Y direction side and on the −Y direction side of the element substrate 18. The ends of the first electrode 42 and the second electrode 44 serve as terminals that are coupled to the FPC 13. The wiring of the FPC 13 and the terminals of the element substrate 18 are fitted and heated, thereby allowing the FPC 13 to be mounted on the ultrasonic element array substrate 14. Other than that, the FPC 13 may be mounted on the ultrasonic element array substrate 14 with an anisotropic conductive film interposed therebetween, or with a resin core bump interposed therebetween.

FIG. 10E and FIG. 11A are views corresponding to the acoustic matching member application step of step S4. As shown in FIG. 10E, an acoustic matching member 46 is applied to the surface of the element substrate 18 on the −Y direction side. As shown in FIG. 11A, the acoustic matching member 46 is applied to the center of the ultrasonic element array substrate 14 in plan view. The shape in which it is applied is elongated in the X direction.

FIG. 11B and FIG. 11C are views corresponding to the lens installation step of step S5. As shown in FIG. 11B, the acoustic lens 16 is installed so as to overlap the ultrasonic element array substrate 14 in step S5. Thus, the lower parts of the spacing members 24 are adhered to the ultrasonic element array substrate 14, and the upper parts thereof are in contact with the acoustic lens 16. In other words, the spacing members 24 are installed in contact with the ultrasonic element array substrate 14 and the acoustic lens 16. The ultrasonic element array substrate 14 and the acoustic lens 16 have the same outer shape as viewed from the Z direction. Accordingly, the ultrasonic element array substrate 14 and the acoustic lens 16 can be positioned by fitting their outer shapes.

FIG. 11C is a view with the acoustic lens 16 omitted. As shown in FIG. 11C, when the acoustic matching member 46 is sandwiched by the ultrasonic element array substrate 14 and the acoustic lens 16, the acoustic matching member 46 flows toward the outer circumference. The portion surrounded by the dashed line in the figure is where the acoustic matching member 46 is applied. The arrows indicate the directions in which the acoustic matching member 46 flows. The spacing members 24 are installed at intervals. The spacing members 24 constitute a flow path through which the acoustic matching member 46 flows. Accordingly, the acoustic matching member 46 can flow from the center of the ultrasonic element array substrate 14 toward the outer circumference.

The shape of the spacing members 24 is circular or elliptical in plan view as viewed from the −Z direction. Such a circular or elliptical shape has no corners, thereby allowing a fluid to flow with a low resistance along its outer circumference. Accordingly, when the acoustic matching member 46 is allowed to flow in the location where the spacing members 24 are present, the acoustic matching member 46 moves along the spacing members 24. At this time, the acoustic matching member 46 pushes out the air located in the space between the ultrasonic element array substrate 14 and the acoustic lens 16, and therefore the intervals of the spacing members 24 can be filled tightly with the acoustic matching member 46.

The acoustic matching member 46 overflowing from the space between the ultrasonic element array substrate 14 and the acoustic lens 16 may be removed with a spatula or the like. It is also possible to adjust the amount of the acoustic matching member 46 to be applied so that the acoustic matching member 46 does not overflow from the space between the ultrasonic element array substrate 14 and the acoustic lens 16.

FIG. 11D is a view corresponding to the acoustic matching member solidification step of step S6 and the frame installation step of step S7. As shown in FIG. 11D, the acoustic matching member 46 is heated and dried so as to serve as the acoustic matching unit 15 in step S6. A material that solidifies by reaction with light or a material that solidifies by reaction with moisture may be used for the acoustic matching member 46.

An adhesive material is applied to the outer side surface of the inner frame 34 in step S7. Next, the inner frame 34 is inserted from the −Z direction side so as to fit the ultrasonic element array substrate 14 and the acoustic lens 16. Next, the outer frame 35 is inserted from the Z direction side to fit the inner frame 34. Next, the adhesive material between the inner frame 34 and the outer frame 35 is solidified so that the inner frame 34 and the outer frame 35 are adhered to each other. At this time, it is preferable that a load is applied in a manner such that the inner frame 34 and the outer frame 35 sandwich the ultrasonic element array substrate 14 and the acoustic lens 16. This allows the ultrasonic element array substrate 14 and the acoustic lens 16 to be fixed with an accurate spacing therebetween. By performing the aforementioned steps, the ultrasonic device 9 is achieved.

As described above, this embodiment has the following effects.

(1) According to this embodiment, the acoustic lens 16 is used in contact with the material being examined. At this time, the acoustic lens 16 is pressed by the material being examined. Stress occurs inside the acoustic lens 16. The acoustic matching unit 15 made of resin, which is susceptible to deformation, deforms due to the stress of the acoustic lens 16. On the other hand, the columnar spacing members 24 are in contact with the acoustic lens 16 and the ultrasonic element array substrate 14 so as to transfer the stress of the acoustic lens 16 to the ultrasonic element array substrate 14. Thus, the thickness of the acoustic matching unit 15 is kept constant, thereby suppressing the deformation of the acoustic lens 16, so that ultrasound can be accurately focused. Further, ultrasound reflected by the material being examined also can be accurately focused on the ultrasonic element 36 since the deformation of the acoustic lens 16 is suppressed. As a result, the ultrasonic device 9 can transmit and receive ultrasound efficiently.

(2) According to this embodiment, the spacing members 24 are installed in locations that do not overlap with the ultrasonic elements 36. Accordingly, the ultrasonic elements 36 are overlapped by the ultrasonic matching unit 15 formed of resin. Accordingly, the ultrasonic device 9 can emit ultrasound with an acoustic impedance adjusted by the acoustic matching unit 15. Further, in the ultrasonic device 9, the acoustic matching unit 15 can adjust the acoustic impedance of the received ultrasound and emit it to the ultrasound element 36.

(3) According to this embodiment, the shape of the spacing members is circular or elliptical. A circle or an ellipse has no corners, thereby allowing a fluid to flow along its outer circumference with little resistance. Accordingly, when the acoustic matching member 46 is allowed to flow in the location where the spacing members 24 are present, the acoustic matching member 46 moves along the arrangement of the spacing members 24. At this time, the acoustic matching member 46 pushes out the air located in the space between the ultrasonic element array substrate 14 and the acoustic lens 16, and therefore the intervals between the spacing members 24 can be filled tightly with the acoustic matching member 46.

### Second Embodiment

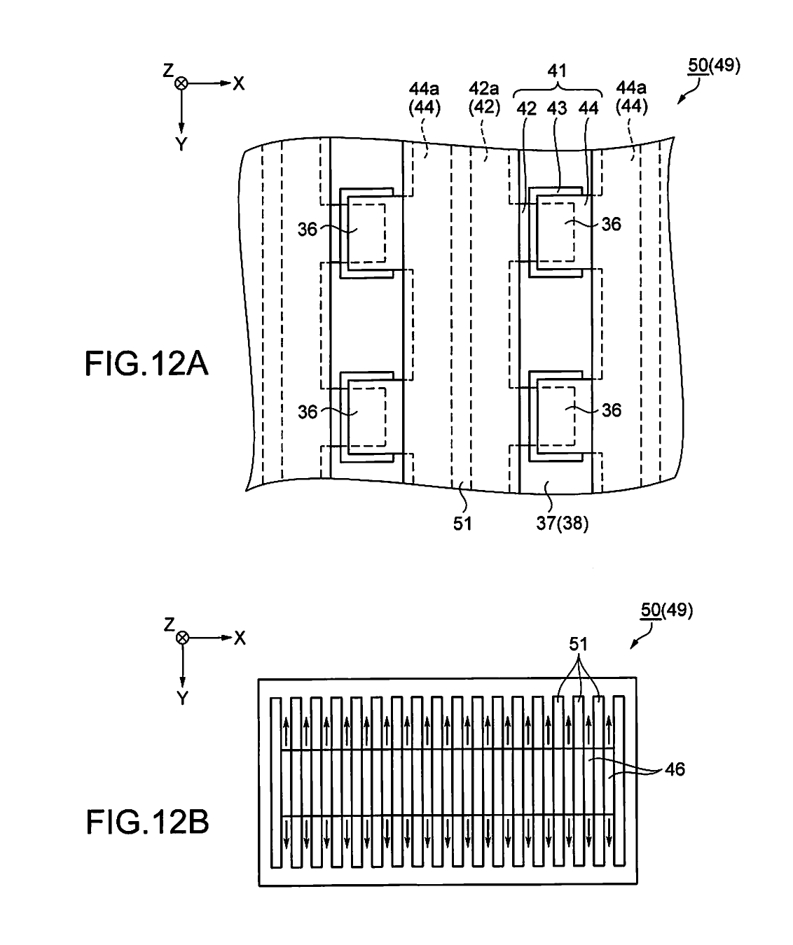

Next, an embodiment of an ultrasonic device will be described with reference to FIGS. 12A and 12B. FIG. 12A is a schematic plan view showing a main part of a configuration of the ultrasonic element, with the acoustic lens 16 omitted and with the spacing members installed. FIG. 12B is a schematic plan view showing a configuration of the ultrasonic element array substrate 14, with the spacing members and the acoustic matching unit installed. In these figures, the FPC 13 is omitted. This embodiment is different from the first embodiment in that a shape of spacing members that is different from that of the spacing members 24 shown in FIGS. 7A and 7B is employed. It should be noted that descriptions for the same parts as the first embodiment are omitted.

In this embodiment, the ultrasonic device 49 includes an element substrate 50, as shown in FIGS. 12A and 12B. The element substrate 50 includes the base substrate 37 on which the vibrating membrane 38 is installed. On the vibrating membrane 38, the first electrode 42 and the second electrode 44 are installed. On the upper side of the first line 42a and the second line 44a, a spacing member 51 is installed so as to cover the first line 42a and the second line 44a. The spacing member 51 has the same function as in the first embodiment, and the spacing member 51 maintains the thickness of the acoustic matching unit 15 constant.

The spacing member 51 hardly allows ultrasound to pass therethrough, and is arranged between ultrasonic elements that are adjacent to each other in the X direction, so as to extend in the form of a wall. It is difficult for ultrasound to pass through the spacing member 51, and the spacing member 51 regulates the direction in which ultrasound propagates. Accordingly, it is possible to suppress a case in which the ultrasonic elements 36 located in the X direction with the spacing member 51 interposed therebetween influence each other via ultrasound.

The spacing member 51 is formed using a material that has low water permeability and hardly allows moisture to pass therethrough. For example, epoxy resin can be used as a material of the spacing member 51. The spacing member 51 is arranged so as to cover the first line 42a and the second line 44a. Accordingly, the spacing member 51 suppresses a case in which moisture attaches to the first line 42a and the second line 44a, and thus can prevent the galvanic corrosion of the first line 42a and the second line 44a.

As shown in FIG. 12B, spacing members 51 are installed on the element substrate 50 in the spacing member forming step of step S2. In the acoustic matching member application step of step S4, the acoustic matching member 46 is applied. In the lens installation step of step S5, the acoustic matching member 46 is sandwiched by the element substrate 50 and the acoustic lens 16. At this time, the acoustic matching member 46 is pressed by the element substrate 50 and the acoustic lens 16, so as to flow toward the outer circumferential side.

The spacing members 51 constitute flow paths through which the acoustic matching member 46 flows. The acoustic matching member 46 moves along the spacing members 51, and therefore pushes out air bubbles, thereby allowing the intervals of the spacing members 51 to be filled tightly with the acoustic matching member 46.

As described above, this embodiment has the following effects.

(1) According to this embodiment, the spacing member 51 is arranged between the ultrasonic elements 36, so as to extend in the form of a wall. It is difficult for ultrasound to pass through the spacing member 51, and the spacing member 51 regulates the direction in which ultrasound propagates. Accordingly, it is possible to suppress a case in which the ultrasonic elements 36 located with the spacing member 51 interposed therebetween influence each other via ultrasound.

(2) According to this embodiment, the spacing member 51 is arranged so as to cover the first line 42a and the second line 44a. The spacing member 51 has a structure that hardly allows moisture to pass therethrough. Accordingly, the spacing member 51 suppresses a case in which moisture attaches to the first line 42a and the second line 44a, and thus can prevent the galvanic corrosion of the first line 42a and the second line 44a.

(3) According to this embodiment, the spacing members 51 form flow paths through which the acoustic matching member 46 flows. The acoustic matching member 46 moves along the spacing members 51, and thus the air between the spacing members 51 is pushed out by the acoustic matching member 46. As a result, the intervals of the spacing members 51 can be filled tightly with the acoustic matching member 46.

### Third Embodiment

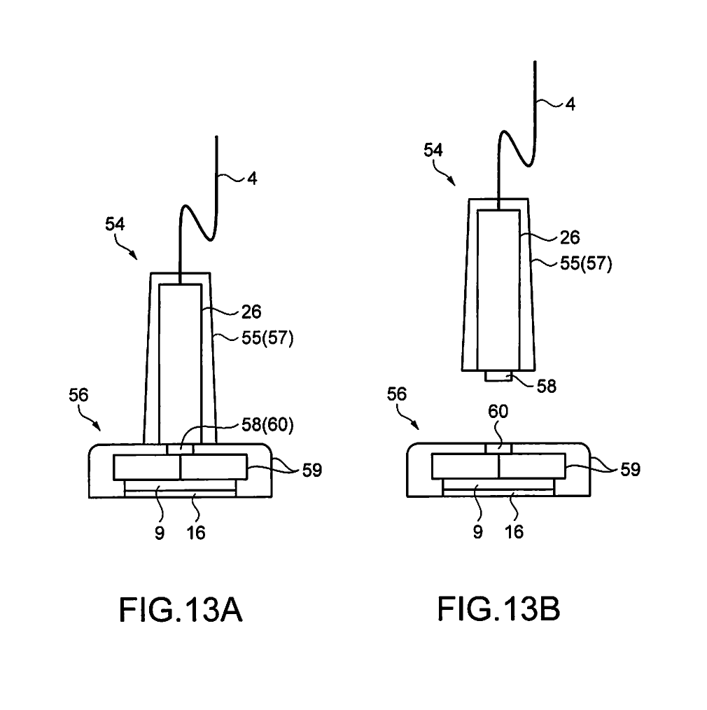

Next, an embodiment of an ultrasonic probe will be described with reference to FIG. 13A and FIG. 13B, which are schematic side views showing a configuration of the ultrasonic probe. This embodiment is different from the first embodiment in that the ultrasonic probe is separable into a body and an ultrasonic probe head. It should be noted that descriptions for the same parts as in the first embodiment are omitted.

As shown in FIG. 13A, an ultrasonic probe 54 includes a probe body 55 and a probe head 56. The probe body 55 includes a body housing 57, and the processing circuit 26 is installed inside the body housing 57. The processing circuit 26 is connected to the apparatus body 2 via the cable 4. A first connector 58 is installed in the body housing 57, and the first connector 58 is connected to the processing circuit 26.

The probe head 56 includes a head housing 59 as a housing, and the ultrasonic device 9 is incorporated in the head housing 59. The acoustic lens 16 of the ultrasonic device 9 is exposed from the head housing 59. A second connector 60 connected to the first connector 58 is installed in the head housing 59, and the processing circuit 26 and the ultrasonic device 9 are electrically connected to each other via the first connector 58 and the second connector 60.

As shown in FIG. 13B, the probe body 55 and the probe head 56 are separable from each other. The first connector 58 and the second connector 60 allow disconnection and connection. A plurality of probe heads 56 are prepared for different frequencies of ultrasound to be transmitted and received by the ultrasonic device 9. Depending on the properties of the material being examined or the depth of the portion of the material being examined, an appropriate probe head 56 can be connected to the probe body 55.

As described above, this embodiment has the following effects.

(1) According to this embodiment, the probe head 56 includes the ultrasonic device 9 and the head housing 59 supporting the ultrasonic device 9. The ultrasonic probe 54 includes the ultrasonic device 9 that appropriately maintains the thickness of the acoustic matching unit 15, and that transmits and receives ultrasound efficiently. Accordingly, it is possible to provide the ultrasonic probe 54 that transmits and receives ultrasound efficiently.

(2) According to this embodiment, the probe head 54 of the ultrasonic probe 56 can be exchanged. Accordingly, it is possible to exchange it with an ultrasonic device 9 that is suitable for the acoustic impedance or the portion of the material being examined.

### Fourth Embodiment

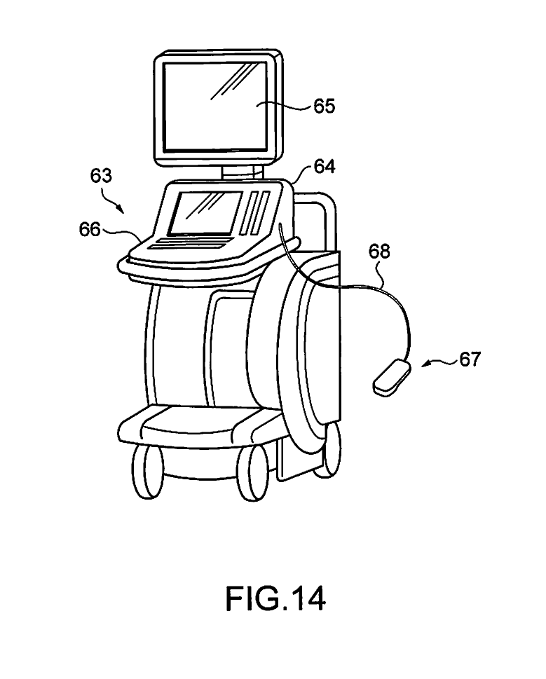

Next, an embodiment of an ultrasonic imaging apparatus will be described with reference to FIG. 14, which is a schematic perspective view showing a configuration of the ultrasonic imaging apparatus. In the ultrasonic imaging apparatus of this embodiment, the ultrasonic probe of the first embodiment is installed. It should be noted that descriptions for the same parts as in the first embodiment are omitted.

As shown in FIG. 14, an ultrasonic imaging apparatus 63 is a mobile ultrasonic imaging apparatus. The ultrasonic imaging apparatus 63 has an apparatus body 64 (electronic apparatus body), a display unit 65 that displays image data to be displayed, a UI unit 66 (user interface unit), an ultrasonic probe 67, and a cable 68. The ultrasonic imaging apparatus 63 can be used for the in-vivo measurement of fat thickness, muscle thickness, bloodstream, bone density, or the like. The ultrasonic device 9 provided in the ultrasonic imaging apparatus 63 appropriately maintains the thickness of the acoustic matching unit 15 and transmits and receives ultrasound efficiently. Accordingly, it can be said that the ultrasonic imaging apparatus 63 is an apparatus provided with the ultrasonic device 9 that transmits and receives ultrasound efficiently.

The invention is not limited to the foregoing embodiments. The specific arrangements and procedures in practicing the invention may be altered by another arrangement or the like as necessary as long as the objects of the invention can be achieved. Many modifications can be made by a person of ordinary skill in the art without departing from the technical scope of the invention. Examples of the modifications will be described below.

Modification 1

In the first embodiment, the spacing members 24 are circular or elliptical. However, there is no limitation on the shape of the spacing members 24. They may be in various forms such as a cone, elliptic cone, cube, rectangular parallelepiped, triangular prism, and polyhedral prism. The shape of the spacing members 24 can be selected so as to facilitate the manufacture thereof.

Modification 2

In the first embodiment, the ultrasonic element 36 performs both the transmission and reception of ultrasound. It is also possible to separate an element that performs the transmission of ultrasound from an element that performs the reception of ultrasound. Further, it is also possible to provide an element that performs the transmission of ultrasound, an element that performs the reception of ultrasound, and an element that performs the transmission and reception of ultrasound. They may be combined depending on the accuracy requirements in the transmission and reception of ultrasound.

In the first embodiment, the piezoelectric layer 43 is a thin film formed using a photolithographic technique. The piezoelectric layer 43 may be of a thick bulk type. Also in this case, the spacing members 24 keep the thickness of the acoustic matching unit 15 constant, which can make the deformation of the acoustic lens 16 difficult, even if the acoustic lens 16 is pressed.

Modification 3

In the second embodiment, the spacing member 51 is in the form of a continuous rectangular parallelepiped that extends in the Y direction so as to cover the first line 42a and the second line 44a. The spacing member 51 may be divided into multiple parts in the Y direction. The acoustic matching member 46 can be allowed to flow so as to fill the spaces between the spacing members 51.

The entire disclosure of Japanese Patent Application No. 2013-223009, filed Oct. 28, 2013 is expressly incorporated by reference herein.

1. An ultrasonic device comprising:
an ultrasonic element array substrate having a plurality of ultrasonic elements configured to perform at least one of transmission and reception of ultrasound;
an acoustic lens configured to focus the ultrasound;
an acoustic matching unit formed using resin, the acoustic matching unit being arranged between the ultrasonic element array substrate and the acoustic lens; and
a plurality of columnar spacing members arranged between the ultrasonic element array substrate and the acoustic lens so as to be in contact with the ultrasonic element array substrate and the acoustic lens.

2. The ultrasonic device according to claim 1, wherein
the spacing members are installed in locations that do not overlap with the ultrasonic elements in plan view, as viewed in a thickness direction of the ultrasonic element array substrate.

3. The ultrasonic device according to claim 1, wherein
the spacing members are arranged between the ultrasonic elements so as to extend in the form of a wall, and hardly allow ultrasound to pass therethrough in an in-plane direction of the ultrasonic array substrate.

4. The ultrasonic device according to claim 3, wherein
the spacing members have lower water permeability than the acoustic matching unit and are arranged so as to cover wiring for transmitting an electrical signal to an ultrasonic element.

5. The ultrasonic device according to claim 3, wherein
a flow path through which material for an acoustic matching unit flows is formed between two of the spacing members.

6. The ultrasonic device according to claim 1, wherein
the shape of the spacing members is circular or elliptical in the plan view.

7. An ultrasonic probe head comprising:
the ultrasonic device according to claim 1; and
a housing configured to support the ultrasonic device.

8. An ultrasonic probe comprising:
the ultrasonic device according to claim 1; and
a driving circuit configured to drive the ultrasonic device.

9. An electronic apparatus comprising:
the ultrasonic device according to claim 1; and
a processing unit connected to the ultrasonic device, the processing unit being configured to generate an image using an output of the ultrasonic device.

10. An ultrasonic imaging apparatus comprising:
the ultrasonic device according to claim 1;
a processing unit connected to the ultrasonic device, the processing unit being configured to perform processing to generate an image using an output of the ultrasonic device; and
a display unit configured to display the image.
